(19) United States
(12) Patent Application Publication
(10) Pub. No.: US 2010/0249495 A1
(43) Pub. Date: Sep. 30, 2010

(54) MULTIPLE-WIDTH BENCH WITH ADJUSTABLE HEIGHT AND INCLINATION

(76) Inventor: Hayrabet Siradag, Meudon (FR)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/742,099

(22) PCT Filed: Nov. 17, 2008
(86) PCT No.: PCT/FR2008/001613

§ 371 Date: May 10, 2010

(30) Foreign Application Priority Data
Nov. 26, 2007 (FR) 0708237

(51) Int. Cl. A61F 5/00

(52) U.S. Cl. 600/38

(57) ABSTRACT

The invention relates to a multiple-width bench with adjustable height and inclination that is intended for the practice of intimate relations between individuals. This is a special stand designed for avoiding the drawbacks of the standard stands. To this end, it proposes and offers the possibility of sitting in a straddle position on a third of its narrow portion, the remaining wide two thirds being used by another subject to lay down. It comprises a multiple-width board (1) mounted on two sets of legs (ESPa) (ESPb) usually used for office armchairs. The use of the legs in a pair, with the mechanisms bereft of their damping spring assemblies, makes it possible to incline the board (1) towards the narrow side (E) thereof and to obtain a plurality of heights relative to the ground, without any steps, instantly.

The invention is related to the sphere of intimate relations, to be more precise, to the practice of the sexual act or other types of intimate gestures between two persons. From here on, we will refer to this concept as “the act”.

It is a special support system studied and designed with the aim of eliminating and avoiding all discomfort associated with support systems meant for this Act, namely, a bed or a sofa. This system may also be used for other types of gestures that a person has for another, in the intimate sphere, such as massage.

The main disadvantages of the systems in the accomplishments of the said act are the lack of comfortable and points of support that can be easily held on to. Depending upon the physical and morphological state of the persons, it is more or less easy to embrace each other and carry out the different steps involved in the act without having to put in an extra effort so that the person lying down does not have to bear the entire weight of the other. In order to hold and withhold different postures of the Act, extra strain is put on the muscles as well as the joints of the upper and lower limbs. For certain positions, the knees bear the weight whereas for others, it is the hands along with forearms or even elbows that serve as points of support, which is not always comfortable and, even painful, sometimes, depending upon the physical condition of the persons. Moreover, when the limbs are used as points of support, they remain dedicated to this task and, consequently, miss out on some real action.

For giving a massage to a person with whom one is intimate, the supports mentioned above have even more disadvantages from the “masseur's” point of view.

Indeed, the person to be massaged is often too low and the “masseur” is obliged to hold skewed and slouched postures that are uncomfortable and even dangerous for the back bone.

There is no support that is specially studied and designed for the practice of this act that takes into account individual difficulties, whether they are due to a state of tiredness or low fitness levels of the persons, and the desire to add variety to the act without putting the body through extra strain.

The invention aims at eliminating all the inconveniences of a traditional support and brings you an item that evokes sensuality and is directly related to it.

It consists of a bench (or an upholstered bench if it is padded and lined) whose planar seating's width varies symmetrically with respect to its longitudinal axis. The width along the ⅔rds of its length is suitable for a person to lie on the back whereas the tapering width of the remaining ⅓rds is ideal for another person to sit astride on the bench. The bench is supported by two legs fixed at either ends, whose heights may be different. Thanks to the height-adjustable legs, the distance between the floor and the bench may be varied and the seating may be inclined towards the narrow end.

This combination of a bench of tapering width with two legs of adjustable heights offers the possibility to at least one adult to sit astride on the narrow part of the bench, with the feet resting on the floor, whilst the other person is lying down on the broader part of the bench. The narrow part of the bench has enough space for the two partners to sit astride.

The bench, whose height can be adjusted, offers a whole range of heights from the ground, in the horizontal as well as inclined positions. The user may adjust the height of the bench using a lever, whilst sitting on it. Once the ideal height is reached, the user just has to release the lever in order to block the bench in the chosen position.

The aim is to give the possibility to at least one partner to be able to sit astride whilst resting the feet on the floor during the Act. This position, in which the user rests on natural points of support (buttocks and feet), frees the hands and arms which can then actively participate in the Act. The person sitting astride does not cast his weight on the person lying down and does not have to struggle to bear his own weight.

The advantage of the inclined positions of the bench is that the angle separating both bodies is reduced, thus allowing both bodies to come together completely, with certain ease.

In case both partners are sitting astride, facing each other, they can embrace each other easily and totally.

When one of the partners is standing next to the bench, s/he can approach the partner lying down from any side without knocking into the edges of the described item, unlike with a bed or a sofa. The legs of the bench are well towards the inside so they are not in the user's way. There is no part or edge that sticks out, that could have restricted the approach to the partner lying down. The body contact is therefore immediate and complete. The height adjustment feature comes in handy to instantly adjust the height of the bench as required.

As the positions to perform the act are multiple and varied, it is impossible to describe all the advantages of the bench in each case.

The essential plus point being the comfort and the ease in practicing the act and, in particular, for persons whose physical condition is diminished due to an accident or to age or for persons with a unusual morphological conditions (height or weight falling out of the usual limits). As the muscles and joints are not put to strain, one can, depending upon the fitness level, find and maintain postures with less effort and more comfort.

The seating of the bench is shaped like a “straight-body” bottle. It has a wide part and a narrow part for two different uses.

The wide part resembles a straight body bottle. It is meant for the upper part of the body of the person lying down. The part whose width goes tapering looks like the “shoulders’ of a bottle. It is useful as clearance space for the knees of the person sitting astride on the narrowest part of the bench, which resembles a bottle-neck. This is meant for sitting astride comfortably without having to open the legs extra wide. Finally, the last part of the bench, which is slightly wider, is like a bottle collar. This part is slightly wider than the neck and offers an alternative, more comfortable and wider surface for sitting down. It is also necessary for fitting in the assembly plate.

The legs mentioned above are the same as the system of legs used in office chairs. It is a system that includes an assembly plate that is fixed under the bench and that allows a certain degree of inclination, a lever-activated glide piston incorporated into the assembly plate and a leg base that supports the base of the glide piston. The assembly plate has a shock-absorber spring system in order to counterbalance the tilting piston. This shock-absorber spring shall be removed from the system in order not to restrict the tilting. In this way, the combined use of these two systems whose shock-absorber springs have been removed, under the same bench, makes it possible to carry out individual adjustment of each of the piston legs, to incline the surface in a certain way, within the limits of the tilting capacity of the assembly plate and the maximum piston stroke distance of the glide pistons used.

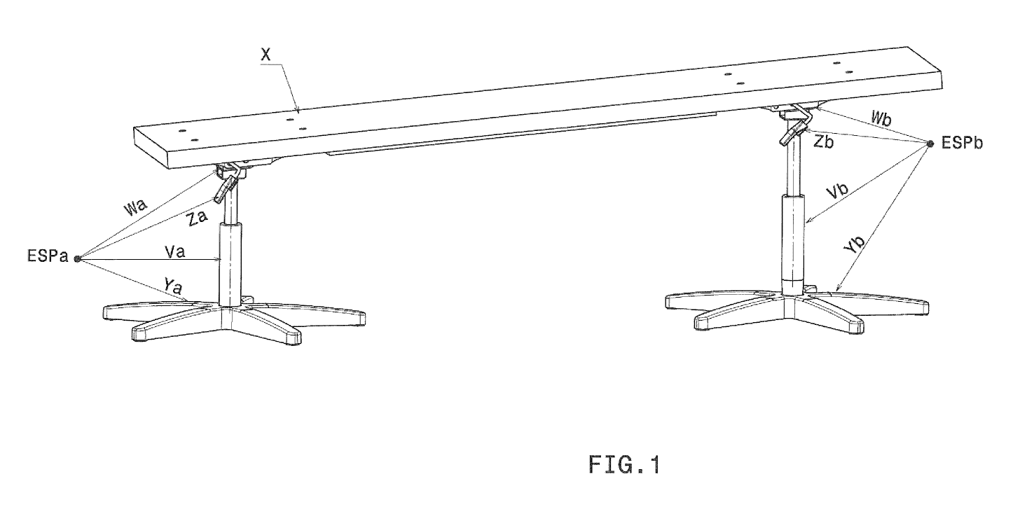

FIG. 1 illustrates this application
Seating laid out (X)
Two sets of leg systems for office chairs (ESPa) (ESPb) and their components
Assembly plates (Wa) (Wb)
Glide pistons (Va) (Vb)
Leg bases (Ya) (Yb)
Command levers (Za) (Zb)

The glide piston (Vb) is set higher than glide piston (Va). In this way, the bench remains inclined towards the leg that is lower. This combination also offers, whether the bench is inclined or not, the possibility of setting the bench at any height from the floor, without there being any pre-defined set heights.

One way of laying out the multi-width bench, at adjustable heights and inclinations and its components is illustrated in the following figures:

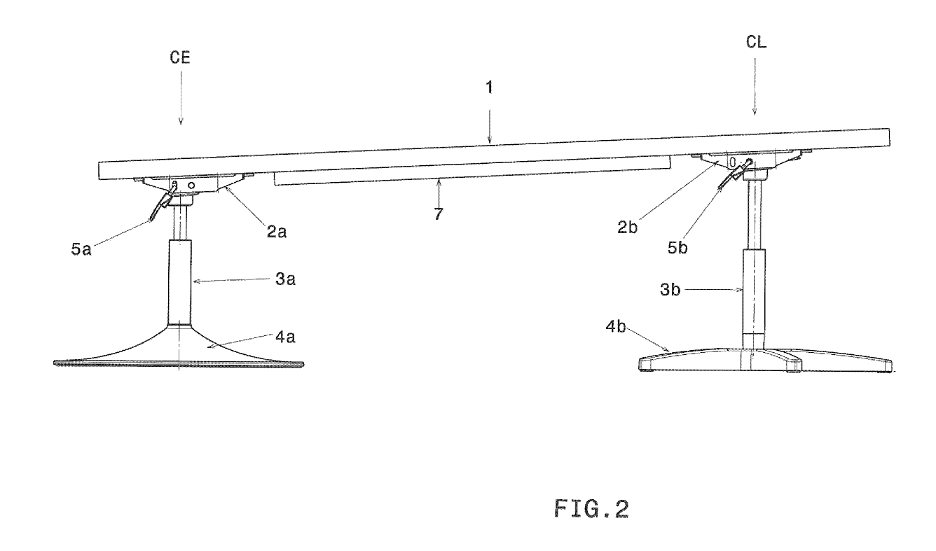

FIG. 2 shows a lateral view of the unit, with the bench (1) in the inclined position, illustrating one assembly plate (2b) fixed close to the edge of the wide end (CL) and the other (2a) close to the edge of the narrow end (CE), the glide pistons (3a) (3b) and the leg bases (4a) (4b). Here, the median supports (7) fixed under the bench are also visible.

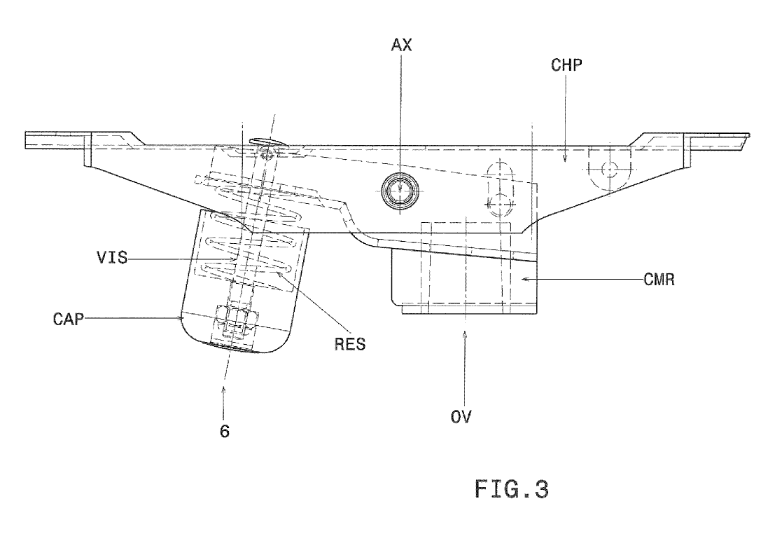

FIG. 3 gives a lateral view of the assembly plate, before the removal of the spring shock-absorber (6) system. It is the state of the assembly plate under normal use conditions under the horizontal bench. The assembly plate used for the multi-width bench has been designed so as to allow tilting towards the back of the seat of the chair in the normal course of its use. This type of mechanism includes, in its main assembly plate (CHP), fixed to the bench seating, a reduced mobility compartment that allows tilting along the axis (AX) which connects these two units, until it hits the stop at one end or another. Here, it is the mobile compartment (CMR) which has an orifice (OV) that holds the head of the glide piston. In this way, depending upon the pressure applied by the force and the sitting position of the user, the seat tilts more or less.

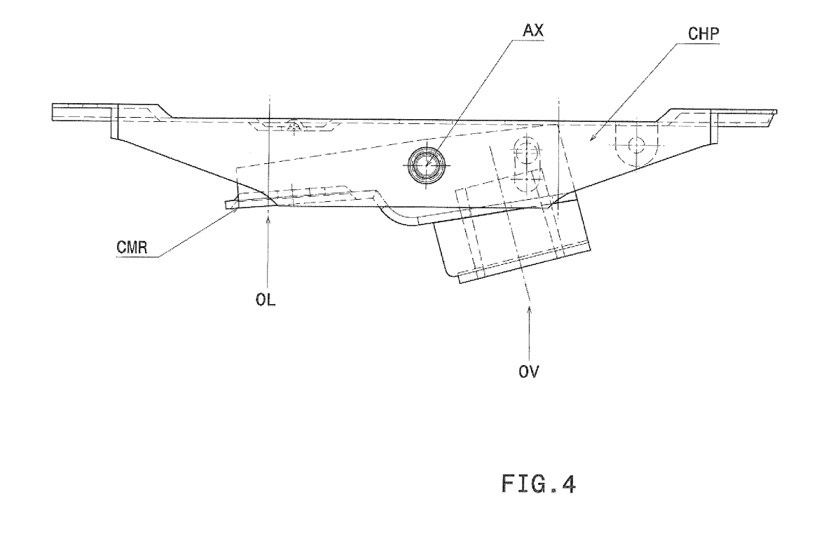

FIG. 4 gives a lateral view of the same plate after the removal of the spring shock-absorber system. In this case, the plate tilts the opposite way in the absence of any restraining force.

The function of the shock-absorber spring system (6) is to stabilize the bench by absorbing the pressure exerted by the user. Its big spring (RES) is held in place by a cap (CAP) that is used to regulate its pressure at the same time. For this, the cap presses on the big screw (VIS) whose base is held by the main assembly plate (CHP) and which crosses the connecting orifice (OL) of the mobile compartment (CMR) forming the spring axis (RES). The screw head (VIS) is fixed under the top of the cap.

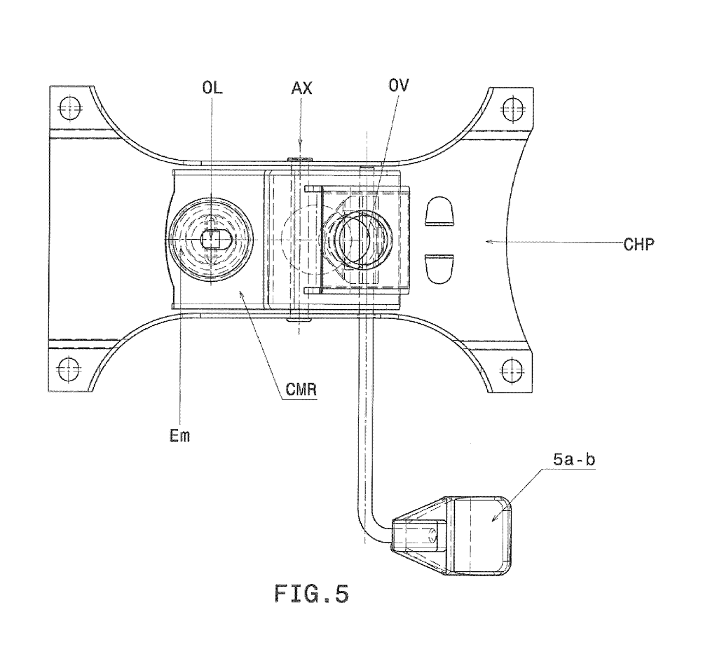

FIG. 5 gives a vertical view of the assembly plate where you can see the slot (Em) for the spring system (6), the connecting orifice (OL) of the axial screw (VIS) as well as the orifice (OV) where the glide piston head is housed.

This capacity to be titled of this type of mechanism is used for inclining the multi-width bench by releasing all strain on the spring (RES). In order to obtain strain-free, immediate and free inclination, the spring effect is eliminated by simply removing the spring. The mobile compartment (CMR) shall then be free to slide around.

The inclination of the bench is obtained due to the difference in heights of the two legs. In case of unequal heights, the bench shall be inclined towards the leg that is lower, by virtue of the inclination of the mobile compartments (CMR) of both assembly plates which are thus freed instantaneously. This is how it is possible to obtain and combine different degrees of inclination depending upon the height of the legs from the floor that varies between the minimum and maximum values of the travel distance of the respective glide pistons (PIS).

The tilting capacity of the mobile compartments (CMR) of the mechanisms allows sufficient angle so that the maximum inclination of the bench, obtained by leaving the maximum difference between the heights of the two glide pistons of the legs, is attained.

Any given inclination of the bench may be maintained in a stable condition thanks to the solidarity that is created between the two leg units (ESPa) (ESPb). Indeed, the removal of a spring system from a assembly plate, habitually used for an office chair, would have made the mechanism unstable due to the effect of the user's weight and movements. However, in this case, the combination of two such leg systems without springs (RES), under the same planar seating, cancels the instability by the solidarity force created between them. In simpler words, they compensate for each other.

The height of the legs may be adjusted by operating the levers (5a) (5b) which are tucked away on the side of the main assembly plate (CHP). By operating them, the user applies pressure on the tip of the glide piston (PIS). At the same time, he puts pressure due to his weight and therefore, the piston goes down. If he annuls the effect of this weight, the piston goes up. By letting go of the lever at the desired height, he stops the movement of the piston and the leg remains at that height. Thus the user may instantly adjust the heights at either ends of the bench and at the same time, obtain the desired inclination.

The mobile compartment (CMR) may only be tilted one way. The mechanisms are assembled in such a way as to allow for an inclination towards the narrow end (CE) of the bench.

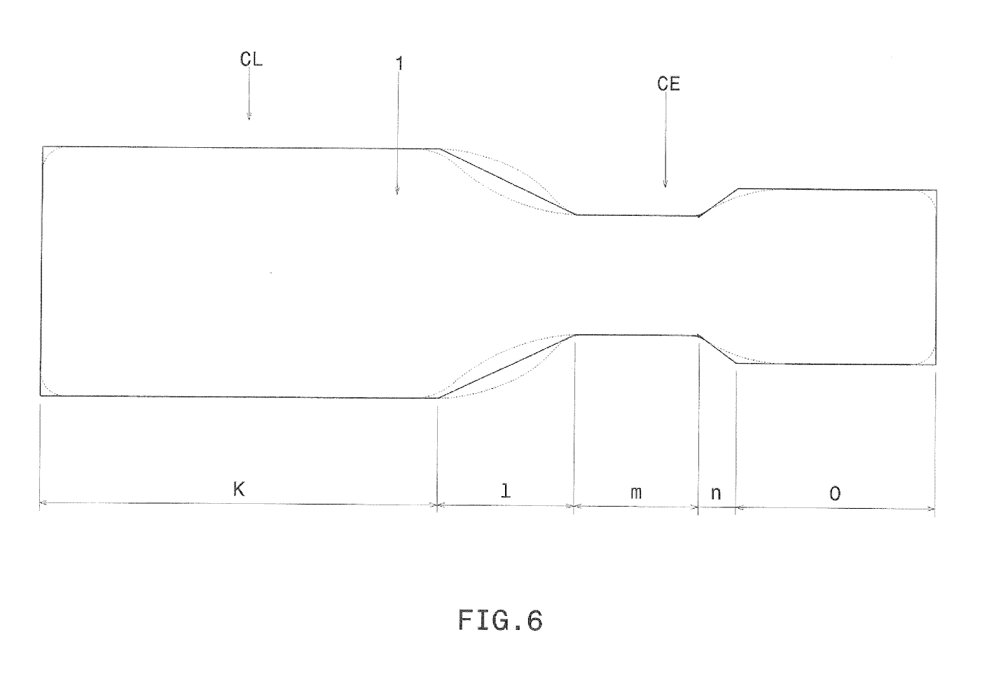

FIG. 6 gives a vertical view of the bench (1), illustrating its “straight body” bottle shape. Its dimensions are as follows, give or take a 15% tolerance.

It is 155 cm long. Over half this length, it has a constant width of 45 cm, forming a rectangle (k). The width goes tapering at the end of this part (k) to attain 22 cm over the next 20 cm of length (l). The bench has the same width over the next 20 cm (m). At the end of this part (m), the bench's width increases gradually over 10 cm to attain a stable width of 30 cm over the remaining 30 cm of length (o). Of course, the narrowing and widening are symmetrical with respect to the longitudinal axis of the bench. The contours of the corners may or may not be rounded off. The bench's contours at places where the width varies may be in the form of straight lines, concave or convex surfaces with some passages that are more or less rounded off.

The parts (k)+(l) of the bench are meant for the body of the person lying down whereas the part (m) is for another person, sitting astride, without having to have the legs wide apart. The part (l) is useful as a clearance area for the knees of the person sitting on the parts (m) and (n). The last part (o) is an alternative seating, which also happens to be the surface needed for assembling the mechanism underneath.

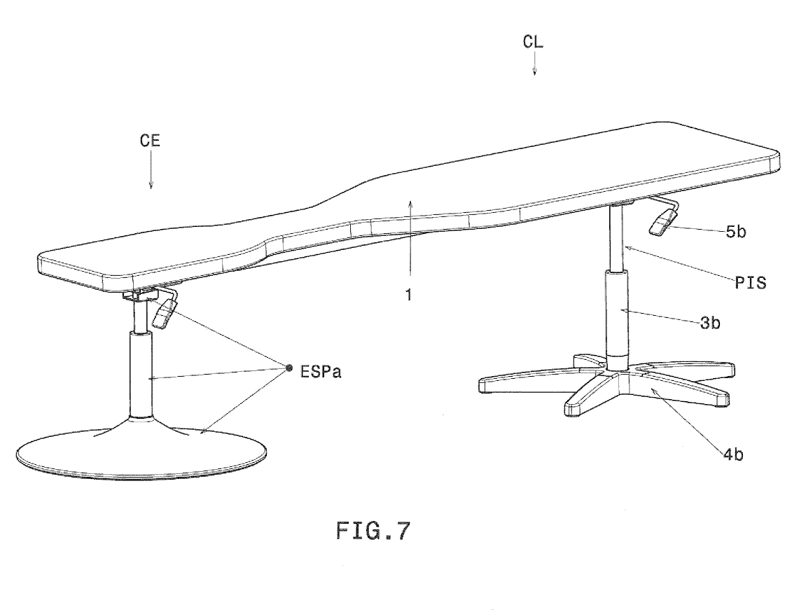

FIG. 7 shows a general view of the bench.

It is clear for people well-versed with this art that this invention makes it possible to lay out the bench in various specific positions without jeopardizing the very purpose for which the invention claims to have been made. Consequently, the present way of laying out the bench is only for illustration sake but may be modified as desired, without losing sight of its purpose, as laid down by the scope of the claims enclosed. The invention must not be limited to the details given above.

1. The multi-width bench with adjustable height and inclination is meant to be used as a support for intimate relations between individuals and features a bottle-shaped planar seating (1) mounted on two leg systems (ESPa) (ESPb) as in an office chair, that can be height-adjusted independently, thus allowing a height difference between them and multiple degrees of inclination towards the narrow end (CE) of the bench and several heights in the inclined position given to the bench (1). All this without discontinuity and without any set heights. It allows at least two adults to sit astride easily over one-third of its length starting from the very end of the narrow part of the bench, whilst providing support on the floor in this position.

2. The multi-width bench with adjustable height and inclination as per claim n^{o} 1 features the combination of two leg systems (ESPa) (ESPb) of an office chair, each of which comprises a mechanism (2a) (2b) allowing tilting, a gas piston (3a) (3b) operated by a lever (5a) (5b) and a leg base ((4a) (4b) that houses the base of the piston, in order to allow multiple heights from the floor, without set intervals, of the bench (1), irrespective of the degree of inclination, within the limits of the angle resulting from the tilting of the assembly plates (2a) (2b) and the maximum distance between the pistons (3a) (3b) respectively.

3. The multi-width bench with adjustable height and inclination as per claim n^{o} 1 and n^{o} 2 features the type of assembly plates (2a) (2b) used, give a tilting capacity, without the spring shock-absorber systems (6) that hinder the tilting movement.

4. The multi-width bench with adjustable height and inclination as per claim n^{o} 1 which includes a planar seating (l) characterized by its “straight-body bottle” shape, the wide part meant for the upper part of the body of the person lying down and, at the same time, its narrow part (m)+(n)+(o), offering another person place to sit astride easily and keeping balance, in the following dimensions, with more or less 20% tolerance: It is 155 cm long. Over half this length, it has a constant width of 48 cm, forming a rectangle (k). The width goes tapering at the end of this part (k) to attain 22 cm over the next 20 cm of length (l). The bench has the same width over the next 20 cm (m). At the end of this part (m), the bench gradually widens over 10 cm length (n) to attain a constant width of 30 cm over the remaining 30 cm of length (o), with passages that are more or less rounded off.

5. The application for two units of leg systems for office chairs (ESPa) (ESPb), each one made up of a tilting assembly plate (Wa) (Wb), the latter being equipped with a lever (Za) (Zb), a glide piston (Va) (Vb) and leg bases (Ya) (Yb), whose specificity lies in the fact that there are two of them that are fitted under the same given bench (X), apart from the use of the multi-width bench with adjustable heights and inclinations as per claims (1), (2) and (3).
